(19) United States
(12) Patent Application Publication
(10) Pub. No.: US 2014/0271492 A1
(43) Pub. Date: Sep. 18, 2014

(54) Manufacturing Process for Effervescent Dosage Forms

(71) Applicant: Mylan Inc., Canonsburg, PA (US)

(72) Inventor: Tammy Bartley, Morgantown, WV (US)

(21) Appl. No.: 14/215,511

(22) Filed: Mar. 17, 2014

Related U.S. Application Data
(60) Provisional application No. 61/790,213, filed on Mar. 15, 2013.

(51) Int. Cl. A61K 31/4468; A61K 9/20

(52) U.S. Cl. CPC A61K 31/4468; A61K 9/2095; A61K 9/2018; A61K 9/2013; A61K 9/2009
USPC 424/43

(57) ABSTRACT

Methods of manufacturing effervescent dosage forms. Methods of manufacturing an effervescent tablet using a dry, direct compression process are disclosed. The methods do not result in the sticking of the mixture to be tableted to the punches during production.

### CROSS-REFERENCE TO RELATED APPLICATIONS

This application claims the benefit under 35 U.S.C. §119(e) of the earlier filing date of U.S. provisional patent application No. 61/790,213 filed on Mar. 15, 2013.

### BACKGROUND OF THE INVENTION

Various formulations for effervescent tablets have been disclosed in U.S. Pat. No. 5,178,878; U.S. Pat. No. 6,200,604; U.S. Pat. No. 8,119,158; U.S. Pat. No. 6,974,590; U.S. Pat. No. 5,223,264; U.S. Pat. No. 5,458,879; EP 1,814,831; US 2011/0281008; U.S. Pat. No. 5,171,571; U.S. Pat. No. 5,817,337; EP 2,515,857; U.S. Pat. No. 6,066,355; U.S. Pat. No. 5,707,654; and U.S. Pat. No. 5,888,544, which are hereby incorporated by reference in their entireties. However, these teach directly mixing the acid and base parts of the effervescent couple, along with other excipients, before tableting. EP 1,945,190 teaches the wet granulation of the acid and the active with silicon dioxide. We found that using a variety of these methods resulted in sticking of the mixture to the tablet punches, which results in a loss of active potency over the course of the run. Alternatively, U.S. Pat. No. 3,577,490 (which is hereby incorporated by reference in its entirety) states that in order to get a tablet which can be manufactured with commercially feasible tableting rates, that the use of Mg stearate and other non-water soluble lubricants must be avoided.

### SUMMARY OF THE INVENTION

The present invention encompasses a method of manufacturing an effervescent tablet using a dry, direct compression process which does not result in the sticking of the mixture to be tableted to the punches.

### DETAILED DESCRIPTION OF THE INVENTION

Initially, a blend [symbol] compress process was evaluated and determined to be unacceptable due to poor compression characteristics of the final blend, in particular sticking. In an effort to improve the processing characteristics, experiments were conducted utilizing a series of blending and milling steps prior to compression. The process of individually blending the effervescent agents (sodium bicarbonate/sodium carbonate and citric acid) with the glidant (silicon dioxide) followed by the milling process, and incorporating the filler (mannitol) and disintegrant (sodium starch glycolate) through blending and milling steps, produced a blend with acceptable flow, density, and tableting characteristics. The processes and formulations of the present invention result in good tablets across all normal operating conditions, including in the higher humidity range of 20-60% relative humidity. As such, the present invention provides a robust method of formulating solid dosage forms that is resilient to traditionally disruptive variables, such as humidity.

The process of coating the acid and/or base components with the glidant protects these agents from ambient moisture as well as from reaction with each other. When either event occurs, the mixture that is in the process of being tableted becomes sticky and gummy. The formulations exemplified here use colloidal silicon dioxide as a coating agent. However, any neutral, non-hygroscopic material with a small enough particle size would function in the same way to evenly coat and protect the acid and/or base component from adventitious reaction with water and/or the complementary half of the effervescent couple. Obvious examples of this include silicon dioxide, talc, and starch. Other examples might include diluents such as cellulose derivatives such as hydroxypropylmethyl cellulose (HPMC), hydroxyethyl cellulose (HEC), hydroxypropyl cellulose (HPC), methyl cellulose, ethyl hydroxyethyl cellulose, starch derivatives such as moderately cross-linked starch; acrylic polymers such as carbomer and its derivatives (Polycarbophyl, Carbopol™, etc.), or microcrystalline cellulose such as Avicel.

Note also that in cases where the acid or base component is not very hygroscopic, then coating of that component is not necessary. See, for examples, some of the following examples where tartaric acid can be used without coating.

The tablet composition itself is fairly straightforward. Appropriate formulation methods are well known to the person skilled in the art: see, for instance, Pharmaceutical Dosage Form: Tablets. Volume 1, 2nd Edition, Lieberman H A et al.; Eds.; Marcel Dekker. New York and Basel 1989, p. 354-356, and literature cited therein, which are hereby incorporated by reference. Suitable additives cited therein comprise additional carrier agents, preservatives, lubricants, gliding agents, disintegrants, flavorings, and dyestuffs.

In addition to the active agent and the glidant, other excipients such as binders, lubricants, humectants, disintegrants, basic agents, acidic agents, sweeteners and the like can be used.

Binder can be selected from, but not limited to, a group comprising ethyl cellulose, gelatine, hydroxy ethyl cellulose, hydroxy methyl cellulose, hydroxypropyl cellulose, hypromellose, magnesium aluminum silicate, methyl cellulose, and povidone.

Lubricant can be selected from, but not limited to, a group comprising calcium stearate, magnesium stearate, polyethylene glycol, PEG6000, polyvinyl alcohol, potassium benzoate, sodium benzoate, sodium stearyl fumarate, and leucine.

Humectant can be selected from, but not limited to, a group comprising anhydrous sodium sulphate, silica gel, and potassium carbonate.

Disintegrant can be selected from, but not limited to, a group comprising carboxymethyl cellulose calcium, carboxymethyl cellulose sodium, microcrystalline cellulose, silicon dioxide, croscarmellose sodium, crospovidone, hydroxypropyl cellulose, methyl cellulose, povidone, magnesium aluminium silicate, starch, and combinations thereof.

Diluent can be selected from, but not limited to, a group comprising calcium carbonate, calcium sulfate, dibasic calcium phosphate, tribasic calcium sulfate, calcium sulfate, microcrystalline cellulose, lactose, magnesium carbonate, magnesium oxide, maltodextrine, maltose, mannitol, sodium chloride, sorbitol, starch, xylitol, and combinations thereof.

The alkaline component of the effervescent couple can be any suitable alkaline effervescent compound, and typically it is an inorganic base (e.g., an alkali metal carbonate) that is safe for human consumption and provides an effective and rapid effervescent disintegration upon contact with water and the acid compound. The alkaline effervescing compound may be selected from the group consisting of carbonate salts, bicarbonate salts, and mixtures thereof. In some embodiments, the alkaline compound is sodium bicarbonate, sodium carbonate anhydrous, potassium carbonate, and potassium bicarbonate, sodium glycine carbonate, calcium carbonate, L-lysine carbonate, arginine carbonate, and combinations thereof. In some embodiments, the alkaline effervescing compound is sodium bicarbonate, potassium bicarbonate, sodium carbonate, or mixtures thereof.

The acid component of the effervescent couple can be any suitable acid for effervescent compositions. Typically, the acid is an organic or mineral acid that is safe for consumption and which provides effective and rapid effervescent disintegration upon contact with water and the alkaline effervescent compound. The acid may be selected from the group consisting of citric acid, tartaric acid, malic acid, fumaric acid, adipic acid, succinic acid, acid anhydrides, related organic acids, and their mixtures. In some embodiments, the acid is citric acid, and especially useful is anhydrous citric acid or tartaric acid.

The acid salt of the composition can be any suitable acid salt or any mixture of suitable salts. Examples of such a suitable acid salt include disodium dihydrogen pyrophosphate, acid citrate salts including mono sodium citrate, and other salts of related organic acids. Combinations thereof are possible. In some embodiments, the acid salt is a salt of citric acid or tartaric acid, and especially useful is monosodium citrate or monosodium tartarate.

### EXAMPLE 1

Initial development studies were conducted using placebo blends. Based on information found in the literature, an effervescent dosage form was manufactured to evaluate tablet physical properties such as hardness, thickness, friability and disintegration time. These blend/compress experiments exhibited marginal compressibility with a maximum hardness of 3.5 kp, resulting in a tablet friability of greater than 1% for these formulations. Additionally, sticking was observed during the compression process. The spray dried mannitol exhibited slightly better compressibility and less sticking than the granular grade of mannitol. These experiments are summarized in the Table 1.

TABLE 1
Placebo Effervescent Dosage Form Experiments

| Experiment number | | | | | | | | |
|---|---|---|---|---|---|---|---|---|
| X08-036 | 1A1 | | 1A2 | | AB1 | | 2B2 | |
| Part I | mg/unit | % | mg/unit | % | mg/unit | % | mg/unit | % |
| Mannitol (spray-dried) | 102 | 51 | 255 | 51 | 255 | 51.0 | | |
| Mannitol (granular) | | | | | | | 255 | 510 |
| Sodium bicarbonate | 42 | 21 | 105 | 21 | 105 | 21 | 105 | 210 |
| Sodium carbonate | 16 | 8 | 40 | 8 | 40 | 8 | 40 | 8.0 |
| Sodium starch | 8 | 4 | 20 | 4 | 20 | 4 | 20 | 4.0 |
| glycolate (explotab/ | | | | | | | | |
| Citric Acid, | 30 | 15 | 75 | 15 | 75 | 15 | 75 | 15.0 |
| Anhydrous | | | | | | | | |
| Magnesium Stearate | 2 | 1 | 5 | 1 | 5 | 1 | 5 | 1.0 |
| (Veg) | | | | | | | | |
| Total Core Weight | 200 | 100 | 500 | 100 | 500 | 100 | 500 | 100 |

| Process | Screen/Blend | Screen/Bag Blend | Screen/Blend | Screen/Blend |
|---|---|---|---|---|
| | (8 quart) | | (4 quart) | (4 quart) |
| Compression/Tablet | | | | |
| properties | | | | |

| Notes | All of Part II | Material was | Repeat of 1A2 | Repeat of 1B1 |
|---|---|---|---|---|
| | including Mag | blended, a sample | using a 4 quart | using Granular |
| | was blended in | was pulled for | blender. Material | Mannitol. |
| | an 8 quart | carver testing. | blended for 10 | Maximum hardness |
| | blender for 15 | The mag was | minutes, mag | 1.2 kp. Filming |
| | minutes. | added and | added blended | occurred on |
| | Tablets | continue to blend. | additional 5 | punches during the |
| | capping off the | Samples were | minutes. | short compression |
| | press. | pulled at several | Maximum | time (1700 tablet |
| | Maximumhard | bland time | hardness 3.5 kp. | batch size) |
| | ness 2.5 kp. | intervals. Carver | No sticking | |
| | Sticking on | Test demonstrated | occurred during | |
| | punches (8000 | a lubricant blend | the short | |
| | tablet batch | time | compression time | |
| | size). | compressibility | (1700 tablet batch | |
| | | correlation. | size) | |

### EXAMPLE 2

In an effort to minimize the observed sticking, a series of experiments were conducted to improve processing characteristics for the effervescent dosage form utilizing a series of blending and milling steps prior to compression. The experiments are summarized in Table 2. It was observed that the citric acid in the presence of the sodium carbonate/sodium bicarbonate resulted in the filming/sticking of the material to the punch faces during compression. By pre-blending the sodium carbonate/sodium bicarbonate with silicon dioxide (Syloid) and passing it through a mill as well as pre-blending the citric acid with silicon dioxide prior to milling, the sticking was eliminated during the compression process.

TABLE 2
Effervescent Dosage Form Blending/Milling Experiments containing Citric Acid

| | Experiment Number X08-36 | | | | | | | |
|---|---|---|---|---|---|---|---|---|
| | 97A2 | | 97B1 | | 97C1 | | 97D1 | |
| | mg/unit | % | mg/unit | % | mg/unit | % | mg/unit | % |
| Part I | | | | | | | | |
| Mannitol (mannogem EZ spray | 98.00 | 49.0 | 98.00 | 49.0 | 98.00 | 49.0 | 98.00 | 49.0 |
| dried) | | | | | | | | |
| Syloid 244FP | 2.00 | 1.0 | 2.00 | 1.0 | 1.00 | 0.5 | 1.40 | 0.7 |
| Sodium Starch Glycolate | 8.0 | 4.0 | 8.0 | 4.0 | 8.0 | 4.0 | 8.0 | 4.0 |
| Sodium Bicarbonate | 42.0 | 21.0 | 42.0 | 21.0 | 42.0 | 21.0 | 42.0 | 21.0 |
| Sodium Carbonate | 18.0 | 9.0 | 18.0 | 9.0 | 18.0 | 9.0 | 18.0 | 9.0 |
| Part II | | | | | | | | |
| Citric Acid granular | 30.0 | 15.0 | 30.0 | 15.0 | 30.0 | 15.0 | 30.0 | 15.0 |
| Syloid 244FP | | | | | 1.00 | 0.5 | 0.60 | 0.3 |
| Part III | | | | | | | | |
| Magnesium Stearate | 2.0 | 1.0 | 2.0 | 1.00 | 2.0 | 1.00 | 2.0 | 1.00 |
| Total Core Weight | 200.0 | 100.0 | 200.0 | 100.0 | 200.0 | 100.0 | 200.0 | 100.0 |

| | | | | |
|---|---|---|---|---|
| Processing Comments: | Premix | Premix | Premix | Premix |
| | carbonates with | carbonates | carbonates | carbonates |
| | syloid. Clean | with syloid | with syloid | with syloid |
| | mill with Part II. | increase | pass through | pass through |
| | Compressed on | mixing time. | mill. Premix | mill. Premix |
| | Hata. Upper | Clean mill | citric acid with | citric acid with |
| | punch faces light | with Part II. | syloid then | syloid then |
| | film lead to | Compressed | clean mill with | clean mill with |
| | picking. | on Hata. After | part II. Some | part II. After |
| | | 60 min run | haze on upper | 60 min run |
| | | punch faces | punch only. | time punch |
| | | had film | | faces clean. |
| | | present but | | Soft sample |
| | | improved from | | punch faces |
| | | 97A2. | | clean. |

### EXAMPLE 3

Additionally an alternative acid was evaluated, summarized in Table 3. It was determined that tartaric acid which is slightly less water soluble than citric acid exhibited less filming/sticking characteristics. It was possible to eliminate the sticking by screening only the sodium carbonate/sodium bicarbonate/silicon dioxide (SYLOID) premix. It was not required to blend the tartaric acid with silicon dioxide.

TABLE 3
Effervescent Dosage Form Blending/Milling Experiments containing Tartaric Acid

| | Experiment Number | | | | | |
|---|---|---|---|---|---|---|
| | 93A1 | | 93B1 | | 93C1 | |
| | mg/unit | % | mg/unit | % | mg/unit | % |
| Part I | | | | | | |
| Mannitol (mannogem EZ spray | 98.00 | 49.0 | 98.00 | 49.0 | 98.00 | 49.0 |
| dried) | | | | | | |
| Syloid 244FP | 2.00 | 1.0 | 2.00 | 1.0 | 2.00 | 1.0 |
| Sodium Starch Glycolate | 8.0 | 4.0 | 8.0 | 4.0 | 8.0 | 4.0 |
| Tartaric Acid | 30.0 | 15.0 | | | | |
| Sodium Bicarbonate | | | 42.0 | 21.0 | 42.0 | 21.0 |
| Sodium Carbonate | | | 18.0 | 9.0 | 18.0 | 9.0 |
| Part II | | | | | | |
| Tartaric Acid | | | 30.0 | 15.0 | 30.0 | 15.0 |
| Sodium Bicarbonate | 42.0 | 21.0 | | | | |
| Sodium Carbonate | 18.0 | 9.0 | | | | |
| Part III | | | | | | |
| Magnesium Stearate | 2.0 | 1.00 | 2.0 | 1.00 | 2.0 | 1.00 |
| Total Core Weight | 200.0 | 100.0 | 200.0 | 100.0 | 200.0 | 100.0 |

| | | |
|---|---|---|
| Premix mannitol | Premix mannitol | Premix carbonates |
| with syloid. | and carbonates | with syloid then |
| Clean mill with | with syloid then | screen. Clean mill |
| Part II. Lower | screen. Clean | with part II. |
| punch faces | mill with Part II. | Punch faces clean |
| shiny. Upper | Upper punch | the entire run. |
| punch faces film | face filming | |
| present. Need to | significantly | |
| blend carbonates | improved. | |
| with syloid then | Remove | |
| screen. | mannitol out of | |
| | premix. | |

### EXAMPLE 4

Studies were conducted to evaluate sorbitol in place of mannitol. It was determined that the sorbitol formulations exhibited increase tablet hardness. Table 4 is a summary of the experiments.

TABLE 4
Effervescent Dosage Form Experiments containing Sorbitol or Mannitol

| | Experiment Number X08-36 | | | | | | | |
|---|---|---|---|---|---|---|---|---|
| | 95A1 | | 96A1 | | 103A1 | | 100A1 | |
| | mg/unit | % | mg/unit | % | mg/unit | % | mg/unit | % |
| Part I | | | | | | | | |
| Fentanyl Citrate | 0.628 | 0.3 | 0.628 | 0.3 | 0.628 | 0.3 | 0.628 | 0.3 |
| Mannitol (mannogem EZ | 94.372 | 46.3 | | | 97.372 | 48.7 | | |
| spray dried) | | | | | | | | |
| Sorbitol | | | 94.372 | 47.2 | | | 97.372 | 48.7 |
| Syloid 244FP | 2.00 | 1.0 | 2.00 | 1.0 | 1.40 | 0.7 | 1.40 | 0.7 |
| Sodium Starch Glycolate | 8.0 | 3.9 | 8.0 | 4.0 | 8.0 | 4.0 | 8.0 | 4.0 |
| Sodium Bicarbondate | 42.0 | 20.6 | 42.0 | 21.0 | 42.0 | 21.0 | 42.0 | 21.0 |
| Sodium Carbonate | 21.0 | 10.3 | 21.0 | 10.5 | 18.0 | 9.0 | 18.0 | 9.0 |
| Part II | | | | | | | | |
| Tartaric Acid | 34.0 | 16.7 | 30.0 | 15.0 | | | | |
| Citric Acid granular | | | | | 30.0 | 15.0 | 30.0 | 15.0 |
| Syloid 244FP | | | | | 0.60 | 0.3 | 0.60 | 0.3 |
| Part III | | | | | | | | |
| Magnesium Stearate | 2.0 | 0.98 | 2.0 | 1.00 | 2.0 | 1.00 | 2.0 | 1.00 |
| Total Core Weight | 204.0 | 100.0 | 200.0 | 100.0 | 200.0 | 100.0 | 200.0 | 100.0 |

| | | | | |
|---|---|---|---|---|
| Tablet Properties | Max hardness | Max hardness | Max hardness | Max hardness |
| | 1.4 kp. | 4.2 kp. | 1.5 kp. | 4.5 kp. |

Formulations 103A1 and 100A1 exhibited acceptable potency, content uniformity, and dissolution assays, and were chosen for further development.

Table 5 discloses a % range of excipients that could be used in Formulations 103A1 and 100A1 m (Table 4):

TABLE 5

| | |
|---|---|
| Target | % range |

| | mg/unit | % | Low | High |
|---|---|---|---|---|
| Mannitol or Sorbitol | 93.372 | 46.686 | 25 | 75 |
| Syloid 244FP | 4 | 2 | 1 | 5 |
| Sodium Starch Glycolate | 8 | 4 | 1 | 10 |
| Sodium Bicarbonate | 42 | 21 | 5 | 30 |
| Sodium Carbonate | 20 | 10 | 3 | 15 |
| Citric Acid granular | 30 | 15 | 5 | 25 |
| Magnesium Stearate | 2 | 1 | 0.5 | 5 |
| Total Core Weight | 200 | 100 | | |

What is claimed is:

1. A method of formulating an effervescent dosage form, the method comprising:
a) individually blending the acid and base components of an effervescent couple with a neutral, non-hygroscopic material to form a pre-blend acid component mixture and a pre-blend base component mixture;
b) combining the pre-blend mixtures from step (a) with a mixture comprising an active pharmaceutical agent and optionally one or more excipients to form a final blend; and
c) forming said final blend from step (b) into the effervescent dosage form.

2. The method of claim 1, wherein the neutral, non-hygroscopic material is a glidant.

3. The method of claim 2, wherein the glidant is selected from the group consisting of silicon dioxide, talc, and starch.

4. The method of claim 1, further comprising milling the pre-blend basic component mixture after blending in step (a) and prior to combining with the active pharmaceutical agent in step (b).

5. The method of claim 1, wherein the optional one or more excipients are selected from the group consisting of diluents, disintegrants, binders, lubricants, humectants, coloring agents, flavoring agents, or mixtures thereof.

6. The method of claim 1, wherein said neutral non-hygroscopic material is selected from the group consisting of hydroxypropylmethyl cellulose, hydroxyethyl cellulose, hydroxypropyl cellulose, methyl cellulose, ethyl hydroxyethyl cellulose, a moderately crosslinked starch, an acrylic polymer, and microcrystalline cellulose.

7. The method of claim 1, wherein the active pharmaceutical ingredient is fentanyl.

8. The method of claim 5, wherein the wherein the diluent is selected from the group consisting of calcium carbonate, calcium sulfate, dibasic calcium phosphate, tribasic calcium sulfate, calcium sulfate, microcrystalline cellulose, lactose, magnesium carbonate, magnesium oxide, maltodextrin, maltose, mannitol, sodium chloride, sorbitol, starch, xylitol, and combinations thereof.

9. The method of claim 5, wherein said disintegrant is selected from the group consisting of carboxymethyl cellulose calcium, carboxymethyl cellulose sodium, microcrystalline cellulose, silicon dioxide, croscarmellose sodium, crospovidone, hydroxypropyl cellulose, methyl cellulose, povidone, magnesium aluminum silicate, starch, and combinations thereof.

10. The method of claim 1, wherein said acid component is selected from the group consisting of organic and mineral acids.

11. The method of claim 10, wherein said acid component is selected from the group consisting of citric acid, tartaric acid, malic acid, fumaric acid, adipic acid, succinic acid, acid anhydrides, related organic acids, and their mixtures.

12. The method of claim 1, wherein said base component is selected from the group consisting of carbonate salts, bicarbonate salts, and mixtures thereof.

13. The method of claim 1, wherein the final blend is formed into the dosage form using compression.

14. A method of preparing an effervescent dosage form, the method comprising the steps of:
a) mixing a base component of an effervescent couple with a first non-hygroscopic material to form a pre-blend base component mixture;
b) milling the pre-blend base component mixture to form a milled base component mixture;
c) mixing an acid component of the effervescent couple with a second non-hygroscopic material to form a pre-blend acid component mixture;
d) milling the pre-blend acid component mixture to form a milled acid component mixture;
e) combining the milled base component mixture from (b) with a mixture comprising an active pharmaceutical agent to form an intermediate mixture;
(d) combining the intermediate mixture with the milled acid component mixture from step (d) to form a final mixture; and
d) forming said final mixture from step (c) into the effervescent dosage form.

15. The method of claim 14, wherein the second non-hygroscopic material is the same as the first non-hygroscopic material in step (a).

16. The method of claim 14, wherein the non-hygroscopic material is selected from the group consisting of silicon dioxide, talc, and starch.

17. A method of preparing an effervescent dosage form, the method comprising:
a) mixing a base component of an effervescent couple with a non-hygroscopic material to form a pre-blend base component mixture.

18. The method of claim 17, further comprising:
b) combining the pre-blend base component mixture from (a) with a mixture comprising an active pharmaceutical agent to form an intermediate mixture;
c) optionally combining the intermediate mixture with an acid component of the effervescent couple to form a final mixture; and
d) forming the final mixture from (c) into the dosage form.

19. A method of preparing an effervescent dosage form, the method comprising:
a) mixing an acid component of an effervescent couple with a non-hygroscopic material to form a pre-blend acid component mixture.

20. The method of claim 19, further comprising:
b) combining the pre-blend acid component mixture from (a) with a mixture comprising an active pharmaceutical agent to form an intermediate mixture;
c) optionally combining the intermediate mixture with a base component of the effervescent couple to form a final mixture; and
d) forming the final mixture from (c) into the dosage form.

21. An effervescent dosage form prepared by the method of claim 1.

22. An effervescent dosage form prepared by the method of claim 17.
